(19) United States
(12) Patent Application Publication
(10) Pub. No.: US 2025/0209856 A1
(43) Pub. Date: Jun. 26, 2025

(54) INFORMATION PROCESSING APPARATUS, INFORMATION PROCESSING SYSTEM, INFORMATION PROCESSING METHOD, AND NON-TRANSITORY COMPUTER-READABLE MEDIUM STORING PROGRAM

(71) Applicant: NEC Corporation, Minato-ku, Tokyo (JP)

(72) Inventors: Misuzu SHINGAI, Tokyo (JP); Katsuya NAKASHIMA, Tokyo (JP); Honami SUZUKI, Tokyo (JP); Takeo TAMURA, Tokyo (JP); Masatoshi SUGISAWA, Tokyo (JP); Shinji KUBOTANI, Tokyo (JP)

(73) Assignee: NEC Corporation, Minato-ku, Tokyo (JP)

(21) Appl. No.: 18/851,907

(22) PCT Filed: May 9, 2023
(86) PCT No.: PCT/JP2023/017484

§ 371 Date: Sep. 27, 2024

(30) Foreign Application Priority Data
May 26, 2022 (JP) 2022-085696

(51) Int. Cl. G06V 40/60; G06F 21/32; G06V 40/10

(52) U.S. Cl. CPC G06V 40/67; G06F 21/32; G06V 40/11

(57) ABSTRACT

An information processing apparatus includes a projection image acquisition unit, a determination unit, and a control signal output unit. The projection image acquisition unit acquires a projection image obtained by projecting a predetermined guidance image onto a hand of a user presented on a biometric information reading apparatus that reads predetermined biometric information from the user's hand. The determination unit determines whether the guidance image and the image of the hand included in the projection image correspond to a predetermined condition. The control signal output unit outputs a predetermined control signal to the biometric information reading apparatus based on the determination.

### TECHNICAL FIELD

The disclosure relates to an information processing apparatus, an information processing system, an information processing method, and a program.

### BACKGROUND ART

Various techniques for reading fingerprints of a user in an immigration inspection or the like are disclosed.

For example, a technique is disclosed in which an arcuate trajectory image that prompts a fingertip not to bulge on a screen along an arcuate trajectory is displayed on the screen as a guidance image (Patent Literature 1).

In addition, a technique of calculating a difference in relative position of a living body with respect to an imaging device when each of a biometric image and a registered image is acquired based on a biometric image generated by imaging the living body as an authentication target and the registered image stored in a registration image storage unit is disclosed (Patent Literature 2).

In addition, a technology is disclosed in which images of at least two or more biological parts of a user are captured, an optimum posture of the user is estimated, and a first posture image representing the estimated posture and a second posture image representing the posture of the biological part captured by an imaging unit are superimposed and displayed (Patent Literature 3).

In addition, a technique is disclosed in which a plurality of marks indicating positions at which a user touches with a finger are displayed on a touch panel, and when it is detected that the user touches the plurality of marks with the finger, biometric information of a hand of the user captured by a biometric sensor is acquired (Patent Literature 4).

### CITATION LIST

### Patent Literature

Patent Literature 1: Japanese Unexamined Patent Application Publication No. 2020-003873
Patent Literature 2: International Patent Publication No. 2010/086993 Patent Literature 3: Japanese Unexamined Patent Application Publication No. 2013-205931
Patent Literature 4: Japanese Unexamined Patent Application Publication No. 2018-128785

### SUMMARY OF INVENTION

However, there is a demand for a technique for more suitably prompting an operation for performing authentication.

In view of the above-described problems, an object of the disclosure is to provide an information processing apparatus or the like that prompts an authentication operation in an easily understandable manner for a user.

An information processing apparatus according to an aspect of the disclosure includes a projection image acquisition unit, a determination unit, and a control signal output unit. The projection image acquisition unit acquires a projection image obtained by projecting a predetermined guidance image onto a hand of a user presented on a biometric information reading apparatus that reads predetermined biometric information from the user's hand. The determination unit determines whether the guidance image and the image of the hand included in the projection image correspond to a predetermined condition. The control signal output unit outputs a predetermined control signal to the biometric information reading apparatus based on the determination.

An information processing method according to an aspect of the disclosure causes a computer to execute the following processing. The computer acquires a projection image obtained by projecting a predetermined guidance image onto the user's hand presented on a biometric information reading apparatus that reads predetermined biometric information from the user's hand. The computer determines whether the guidance image and the image of the hand included in the projection image correspond to a predetermined condition. The computer outputs a predetermined control signal to the biometric information reading apparatus based on the determination.

A program according to an aspect of the disclosure causes a computer to execute the following processing. The computer acquires a projection image obtained by projecting a predetermined guidance image onto the user's hand presented on a biometric information reading apparatus that reads predetermined biometric information from the user's hand. The computer determines whether the guidance image and the image of the hand included in the projection image correspond to a predetermined condition. The computer outputs a predetermined control signal to the biometric information reading apparatus based on the determination.

### BRIEF DESCRIPTION OF DRAWINGS

FIG. 1 is a block diagram illustrating a configuration of an information processing apparatus according to a first example embodiment.

FIG. 2 is a flowchart illustrating an information processing method according to the first example embodiment.

FIG. 3 is a block diagram illustrating a configuration of an information processing system according to a second example embodiment.

FIG. 4 is a block diagram illustrating a configuration of the information processing apparatus according to the second example embodiment.

FIG. 5 is a block diagram illustrating a configuration of an authentication apparatus.

FIG. 6 is a flowchart illustrating an information processing method according to the second example embodiment.

FIG. 7 is a first diagram illustrating a usage example of the information processing apparatus according to the second example embodiment.

FIG. 8 is a view illustrating a guidance image.

FIG. 9 is a second diagram illustrating a usage example of the information processing apparatus according to the second example embodiment.

FIG. 10 is a third diagram illustrating a usage example of the information processing apparatus according to the second example embodiment.

FIG. 11 is a fourth diagram illustrating a usage example of the information processing apparatus according to a third example embodiment.

FIG. 12 is a flowchart illustrating an information processing method according to a fourth example embodiment.

FIG. 13 is a flowchart illustrating an information processing method according to a fifth example embodiment.

FIG. 14 is a first diagram illustrating a usage example of the information processing apparatus according to the fifth example embodiment.

FIG. 15 is a second diagram illustrating a usage example of the information processing apparatus according to the fifth example embodiment.

FIG. 16 is a block diagram illustrating a configuration of an information processing apparatus according to a sixth example embodiment.

FIG. 17 is a flowchart illustrating an information processing method according to the sixth example embodiment.

FIG. 18 is a diagram illustrating a configuration of an information processing apparatus according to a seventh example embodiment.

FIG. 19 is a block diagram illustrating a hardware configuration of a computer.

### EXAMPLE EMBODIMENT

Hereinafter, the disclosure will be described through example embodiments of the invention, but the invention according to the claims is not limited to the following example embodiments. Not all the configurations described in the example embodiments are essential as means for solving the problem. For clarity of description, the following description and drawings are omitted and simplified as appropriate. In each drawing, the same elements are denoted by the same reference numerals, and redundant description is omitted as necessary.

### First Example Embodiment

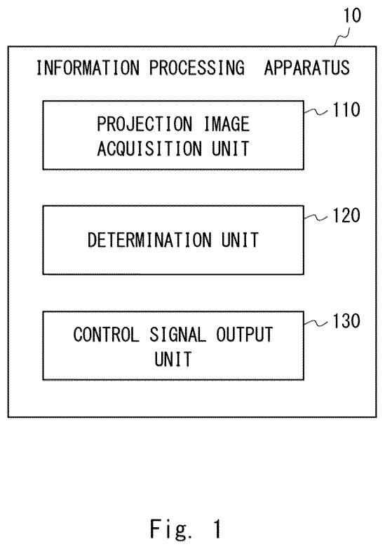

First, a first example embodiment of the disclosure will be described. FIG. 1 is a block diagram illustrating a configuration of an information processing apparatus according to a first example embodiment. The information processing apparatus 10 illustrated in FIG. 1 is used to acquire predetermined biometric information from the user's hand. The predetermined biometric information is, for example, a fingerprint shape pattern, a vein shape pattern, or a combination thereof.

For example, the information processing apparatus 10 acquires the biometric information of the user by cooperating with a biometric information reading apparatus that reads the biometric information of the user who is the user of the information processing apparatus 10. For example, the information processing apparatus 10 is communicably connected to an imaging device that generates image data by imaging a predetermined image, and receives the image data from the imaging device. Furthermore, the information processing apparatus 10 generates a predetermined control signal by processing the received image data, and supplies the generated control signal to the biometric information reading apparatus.

The information processing apparatus 10 may be configured, for example, by a computer, a server, or a dedicated device having a communication function. In the following description, a “computer” may include a server apparatus, a blade, and a cloud computing system. The information processing apparatus 10 mainly includes a projection image acquisition unit 110, a determination unit 120, and a control signal output unit 130.

The projection image acquisition unit 110 acquires the projection image. The projection image is an image obtained by projecting a predetermined guidance image onto the user's hand presented on the biometric information reading apparatus. That is, the projection image includes an image of the user's hand and a guidance image projected on the user's hand. The guidance image guides at least the position of the hand to the user who brings the hand close to the biometric information reading apparatus. The guidance image is set to be projected onto the user's hand when the user brings the user's hand close to the biometric information reading apparatus.

Note that, in the disclosure, the “image” also means image data that is data of the image. That is, the above-described projection image also means “image data of the projection image.” For example, the projection image acquisition unit 110 acquires image data related to the projection image.

The determination unit 120 determines whether the guidance image and the image of the hand included in the projection image correspond to a predetermined condition. The predetermined condition corresponds to, for example, a situation in which the biometric information of the user can be read by the biometric information reading apparatus. Specifically, for example, in a case where the hand is in a preset posture and a guidance image indicating that the biometric information can be read is projected at a predetermined position of the hand, the determination unit 120 can determine that the hand corresponds to the predetermined condition. In addition, in a case where the guidance image indicating that the biometric information can be read is not projected at the predetermined position of the hand, the determination unit 120 may determine that the predetermined condition is not satisfied.

The control signal output unit 130 outputs a predetermined control signal to the biometric information reading apparatus according to the determination. For example, when the determination unit 120 determines that the projection image corresponds to the above-described predetermined condition, the control signal output unit 130 can output a control signal indicating that the biometric information can be read to the biometric information reading apparatus. Alternatively, in this case, the control signal output unit 130 may output a control signal instructing the biometric information reading apparatus to read the biometric information.

Furthermore, for example, in a case where the determination unit 120 determines that the projection image does not correspond to the above-described predetermined condition, the control signal output unit 130 can output a control signal indicating that the biometric information cannot be read to the biometric information reading apparatus. Alternatively, in this case, the control signal output unit 130 may output a control signal instructing not to read the biometric information to the biometric information reading apparatus.

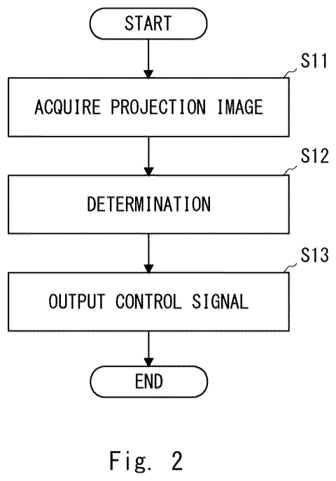

Next, processing executed by the information processing apparatus 10 will be described with reference to FIG. 2. FIG. 2 is a flowchart illustrating an information processing method according to the first example embodiment. The flowchart illustrated in FIG. 2 starts when the information processing apparatus 10 receives a projection image, for example.

First, the projection image acquisition unit 110 acquires a projection image obtained by projecting a predetermined guidance image onto the user's hand presented on the biometric information reading apparatus (step S11). When acquiring the projection image, the projection image acquisition unit 110 supplies the acquired projection image to the determination unit 120.

Next, the determination unit 120 determines whether the guidance image and the hand image included in the received projection image correspond to a predetermined condition (step S12). The determination unit 120 supplies information regarding the determination result to the control signal output unit 130.

Next, the control signal output unit 130 outputs a predetermined control signal to the biometric information reading apparatus based on the above-mentioned determination (step S13). When the control signal output unit 130 outputs the control signal to the biometric information reading apparatus, the information processing apparatus 10 ends a series of processing.

The information processing method executed by the information processing apparatus 10 has been described above. With the above-described configuration, the information processing apparatus 10 can project a guidance image that is intuitively easy for the user to understand on the user's hand, and can guide the user's hand to a predetermined position. Furthermore, in a case where the user's hand exists at a predetermined position, the information processing apparatus 10 can instruct the biometric information reading apparatus to read the biometric information.

The information processing apparatus according to the first example embodiment has been described above. Note that the information processing apparatus 10 includes a processor and a storage device as components not illustrated. The storage device included in the information processing apparatus 10 includes, for example, a storage device including a non-volatile memory such as a flash memory or a solid state drive (SSD). In this case, the storage device included in the information processing apparatus 10 stores a computer program (hereinafter, the program is also simply referred to as a program) for executing the above-described image processing method. In addition, the processor reads the computer program from the storage device into a buffer memory such as a dynamic random access memory (DRAM), and executes the program.

Each component included in the information processing apparatus 10 may be realized by dedicated hardware. Some or all of the components may be implemented by general-purpose or dedicated circuitry, a processor, or the like, or a combination thereof. These components may be configured with a single chip or may be configured with a plurality of chips connected via a bus. Some or all of components of each apparatus may be implemented by a combination of the above-described circuit or the like and a program. Furthermore, as the processor, a central processing unit (CPU), a graphics processing unit (GPU), a field-programmable gate array (FPGA), or the like may be used. Note that the description regarding the configuration described here can also be applied to other apparatuses or systems described below in the disclosure.

Furthermore, in a case where some or all of the components of the information processing apparatus 10 is realized by a plurality of information processing apparatuses, circuits, and the like, the plurality of information processing apparatuses, circuits, and the like may be arranged in a centralized manner or in a distributed manner. For example, the information processing apparatuses, the circuits, and the like may be implemented in the form of a client server system, a cloud computing system, or the like in which they are connected to each other via a communication network. The function of the information processing apparatus 10 may be provided in a software as a service (SaaS) format. In addition, the above-described method may be stored in a computer-readable medium to cause a computer to execute the method.

According to the present example embodiment, it is possible to provide an information processing apparatus or the like that prompts the user to perform the authentication operation in an easily understandable manner.

### Second Example Embodiment

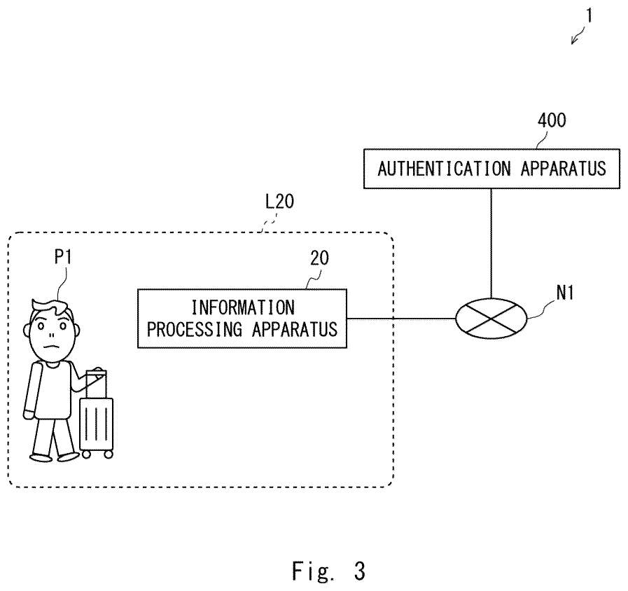

Next, an information processing system will be described with reference to FIG. 3. FIG. 3 is a diagram illustrating an overall configuration of the information processing system 1 according to the second example embodiment. The information processing system 1 is a system that performs biometric authentication of a user who uses an airport, a port, or the like, for example. The information processing system 1 mainly includes an information processing apparatus 20 and an authentication apparatus 400. The information processing apparatus 20 and the authentication apparatus 400 are communicably connected to each other via a network N1. Note that the network N1 may be a telephone line, a wide area network, or a local area network.

The information processing apparatus 20 is installed at a predetermined airport L20, for example, and acquires biometric information of the user P1 who is a passenger. The information processing apparatus 20 that has acquired the biometric information of the user P1 supplies the acquired biometric information to the authentication apparatus 400. When receiving the biometric information from the information processing apparatus 20, the authentication apparatus 400 authenticates the received biometric information. The authentication apparatus 400 that has performed the authentication supplies information regarding a result of the authentication to the information processing apparatus 20. The information processing apparatus 20 that has received the information regarding the result of the authentication from the authentication apparatus 400 executes processing such as passage permission of the user P1 according to the information.

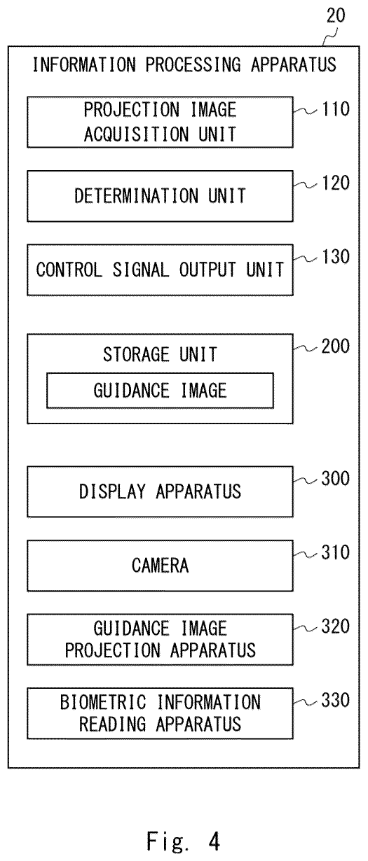

Next, the information processing apparatus 20 will be further described with reference to FIG. 4. FIG. 4 is a block diagram illustrating a configuration of the information processing apparatus 20 according to the second example embodiment. The information processing apparatus 20 mainly includes a projection image acquisition unit 110, a determination unit 120, a control signal output unit 130, a storage unit 200, a display apparatus 300, a camera 310, a guidance image projection apparatus 320, and a biometric information reading apparatus 330. These configurations included in the information processing apparatus 20 are communicably connected as appropriate in order for the information processing apparatus 20 to exhibit the functions described in the disclosure.

The projection image acquisition unit 110 according to the present example embodiment acquires image data regarding a projection image generated by imaging by the camera 310.

The determination unit 120 according to the present example embodiment determines whether the guidance image and the image of the hand included in the projection image correspond to a predetermined condition. The predetermined condition is, for example, that a trigger image indicating the start of reading biometric information in the guidance image is projected on the hand of the user P1.

In addition, the determination unit 120 according to the present example embodiment determines that the projection image includes an image of a hand. In a case where it is determined that the projection image includes the image of the hand, the determination unit 120 supplies information indicating the determination result to the control signal output unit 130.

In a case where the projection image corresponds to the above-described predetermined condition, the control signal output unit 130 according to the present example embodiment outputs a control signal for starting the reading of the biometric information to the biometric information reading apparatus 330.

In addition, the control signal output unit 130 outputs a control signal instructing the guidance image projection apparatus 320 to project a guidance image under a predetermined situation. The predetermined situation is, for example, a case where, in a state before the guidance image projection apparatus 320 projects the guidance image, information indicating a result of determination that the projection image includes an image of a hand is received from the determination unit 120. As a result, the information processing apparatus 20 can start the projection of the guidance image in a case where the user P1 brings his/her hand close to the biometric information reading apparatus 330.

The storage unit 200 is a storage device including a nonvolatile memory such as a flash memory. The storage unit 200 stores at least image data of the guidance image projected by the guidance image projection apparatus 320. Furthermore, the storage unit 200 can store a program or the like of an information processing method executed by the information processing apparatus 20.

The display apparatus 300 is an information display apparatus including liquid crystal, organic electroluminescence, or the like, and notifies the user P1 of the result of authentication, for example. Furthermore, the display apparatus 300 may notify the user P1 of information such as presenting a hand.

The camera 310 is an imaging device that images the hand of the user P1 presented to the biometric information reading apparatus 330 and the guidance image projected on the hand of the user P1. The camera 310 includes an objective lens, an imaging element, an image processing apparatus, and the like. The camera 310 may have a function of zooming, tilting, and panning.

The guidance image projection apparatus 320 projects a predetermined guidance image on the hand of the user P1 for the purpose of guiding the hand of the user P1 to a predetermined position. The guidance image projection apparatus 320 includes, for example, a light source such as a light-emitting diode (LED) or a lamp, an image generation unit that includes a liquid crystal panel, a galvanic mirror, or the like and receives the light source to generate a guidance image, and a projection unit that includes a lens or the like for projecting the image generated by the image generation unit. For example, the guidance image projection apparatus 320 receives a control signal including a predetermined instruction from the control signal output unit 130, and projects a guidance image according to the received control signal.

The biometric information reading apparatus 330 performs a biometric information reading operation according to a control signal received from the control signal output unit 130 and reads predetermined biometric information. The biometric information reading apparatus 330 is provided at a position where biometric information can be read from a hand corresponding to a predetermined condition. The biometric information reading apparatus 330 includes, for example, an image sensor such as an imaging element for reading predetermined biometric information.

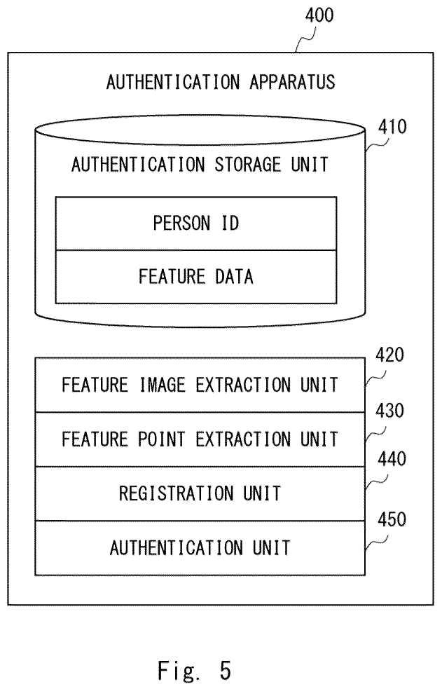

A configuration of the authentication apparatus 400 will be described with reference to FIG. 5. FIG. 5 is a block diagram of the authentication apparatus 400. The authentication apparatus 400 is a computer having an authentication function to be described later. The authentication apparatus 400 receives an image related to the biometric information of the user P1 from the information processing apparatus 20, and extracts a predetermined feature image from the received image to authenticate the person. The feature image is, for example, a fingerprint pattern image. The authentication apparatus 400 mainly includes an authentication storage unit 410, a feature image extraction unit 420, a feature point extraction unit 430, a registration unit 440, and an authentication unit 450.

The authentication storage unit 410 stores a person ID related to a person registered in advance and feature data of the person in association with each other. The feature image extraction unit 420 detects a feature region included in an image relating to biometric information of the user P1 and outputs the feature region to the feature point extraction unit 430. The feature point extraction unit 430 extracts a feature point from the feature region detected by the feature image extraction unit 420, and outputs data regarding the feature point to the registration unit 440. The data relating to feature points is a set of extracted feature points.

The registration unit 440 newly issues a person ID when registering the feature data. The registration unit 440 registers the issued person ID and the feature data extracted from the registered image in the authentication storage unit 410 in association with each other. The authentication unit 450 collates the feature data extracted from the image relating to the biometric information of the user P1 with the feature data in the authentication storage unit 410. In a case where there is feature data that matches the biometric information of the user P1, the authentication unit 450 determines that the authentication has succeeded. On the other hand, in a case where there is no feature data that matches the biometric information of the user P1, the authentication unit 450 determines that the authentication has failed. The authentication unit 450 supplies information regarding success or failure of authentication to the information processing apparatus 20. In addition, in a case where the authentication is successful, the authentication unit 450 specifies a person ID associated with the successful feature data and notifies the information processing apparatus 20 of an authentication result including the specified person ID.

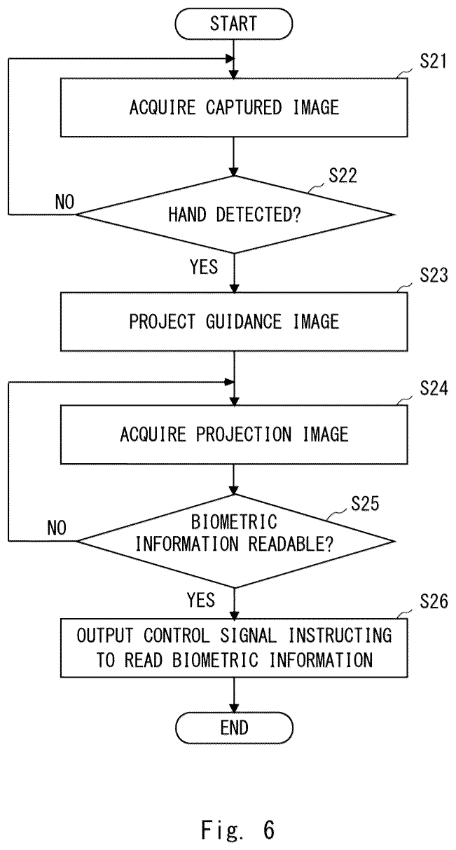

Next, processing executed by the information processing apparatus 20 will be described with reference to FIG. 6. FIG. 6 is a flowchart illustrating an information processing method according to the second example embodiment. The flowchart illustrated in FIG. 6 starts, for example, when the camera 310 of the information processing apparatus 20 starts imaging.

First, the projection image acquisition unit 110 acquires a captured image from the camera 310 (step S21). When acquiring the captured image, the projection image acquisition unit 110 supplies the acquired captured image to the determination unit 120.

Next, the determination unit 120 determines whether an image of the hand of the user P1 is included in the received captured image, in other words, whether the hand is detected (step S22). In a case where it is determined that the hand is not detected (step S22: NO), the information processing apparatus 20 returns to Step S21. On the other hand, when it is determined that a hand has been detected (step S22: YES), the information processing apparatus 20 proceeds to step S23.

Next, in step S23, the control signal output unit 130 supplies a control signal instructing the guidance image projection apparatus 320 to project a guidance image, and the guidance image projection apparatus 320 starts projection of the guidance image in response to the control signal (step S23).

Next, the projection image acquisition unit 110 acquires the projection image captured by the camera 310 (step S24). The projection image acquisition unit 110 supplies the acquired projection image to the determination unit 120.

Next, the determination unit 120 determines whether the guidance image and the image of the hand included in the received projection image correspond to a predetermined condition. More specifically, the determination unit 120 determines whether the biometric information is readable from the received projection image (step S25). When not determining that the biometric information is readable from the received projection image (step S25: NO), the determination unit 120 returns to step S24 and acquires the projection image again. That is, while the determination unit 120 repeats steps S24 and S25, the user P1 attempts to move the hand to a position where the biometric information reading apparatus can read the biometric information according to the projected guidance image. When determining that the biometric information is readable from the received projection image (step S25: YES), the determination unit 120 proceeds to step S26. At this time, the determination unit 120 supplies information regarding the determination result to the control signal output unit 130.

Next, the control signal output unit 130 outputs a control signal instructing the biometric information reading apparatus to read the biometric information based on the above-mentioned determination (step S26). When the control signal output unit 130 outputs the control signal to the biometric information reading apparatus, the information processing apparatus 20 ends a series of processing.

Although the processing executed by the information processing apparatus 20 has been described above, the processing executed by the information processing apparatus 20 is not limited to the above-described processing. For example, the information processing apparatus 20 may set a time limit in a case where steps S24 and S25 are repeated, and may execute processing of outputting an error message to the display apparatus 300 in a case where it is not determined that the predetermined condition is satisfied within the set time limit. Furthermore, in a case where step S24 and step S25 are repeated, the information processing apparatus 20 may set an upper limit of the number of times of repetition, and may execute processing of outputting an error message to the display apparatus 300 in a case where the number of times of not determining to correspond to the predetermined condition reaches the set upper limit.

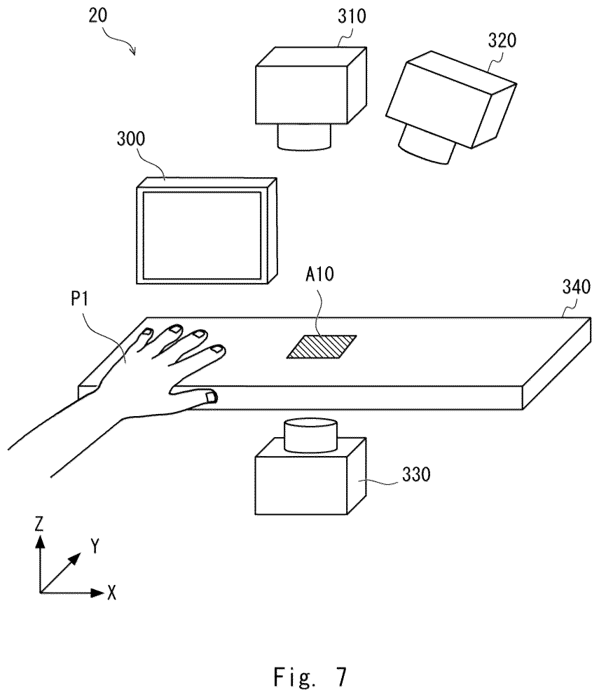

Next, a usage example of the information processing apparatus 20 will be described with reference to FIG. 7. FIG. 7 is a first diagram illustrating a usage example of the information processing apparatus 20 according to the second example embodiment. The information processing apparatus 20 illustrated in FIG. 7 further includes a guide portion 340 in addition to the above-described configuration. The information processing apparatus 20 illustrated here reads the fingerprint pattern of the index finger of the left hand of the user P1 as the biometric information.

Note that a right-handed orthogonal coordinate system is attached to FIG. 7 for convenience of explaining the positional relationship of the components. In the following drawings, when an orthogonal coordinate system is attached, the X-axis, Y-axis, and Z-axis directions in FIG. 7 and the X-axis, Y-axis, and Z-axis directions of the orthogonal coordinate system coincide with each other.

The guide portion 340 has a horizontal main surface. For example, the information processing apparatus 20 guides the user P1 to put out the left hand above the guide portion 340 with the back of the hand facing upward. The guide portion 340 has a readable region A10.

The biometric information reading apparatus 330 is disposed below the readable region A10. That is, when the user's hand or finger is located in the readable region A10, the biometric information reading apparatus 330 can read the biometric information of P1.

The guide portion 340 is, for example, a transparent plate-like member, and is made of glass, acrylic, polycarbonate, or the like. A region of the guide portion 340 excluding readable region A10 may not be transparent. In the guide portion 340, for example, the readable region A10 may be transparent, and the other portions may have a color tone such as black or white. For example, in a case where the other portion is black, the guide portion 340 can absorb the light of the guidance image projected by the guidance image projection apparatus 320. On the other hand, when the other part is white, the guide portion 340 can reflect the light of the guidance image projected by the guidance image projection apparatus 320.

The camera 310 and the guidance image projection apparatus 320 are disposed above the guide portion 340. The camera 310 is installed so as to be able to image the guide portion 340 including the readable region A10 from above the guide portion 340. The guidance image projection apparatus 320 projects a predetermined guidance image from the upper side to the lower side of the guide portion 340.

The display apparatus 300 is provided at a position where the user P1 can visually recognize information to be displayed. For example, the display apparatus 300 is provided near the guide portion 340. As a result, the information processing apparatus 20 can notify the user P1 presenting a hand of predetermined information. Furthermore, for example, the display apparatus 300 may present predetermined information to the user P1 in a case where no guidance image is projected on the hand of the user P1.

In addition, the display apparatus 300 may be realized by the guidance image projection apparatus 320 projecting predetermined information on the surface of the guide portion 340. Alternatively, the guide portion 340 may include the display apparatus 300. That is, for example, the guide portion 340 may include a liquid crystal panel or the like in at least a part thereof.

With the above-described configuration, the information processing apparatus 20 can suitably project the guidance image on the hand presented by the user P1 and guide the hand of the user P1 to the readable region A10. Note that, in the above-described configuration, the information processing apparatus 20 may guide the hand of the user P1 by the guide portion 340 coming into contact with the hand of the user P1. Furthermore, the information processing apparatus 20 may be configured such that the guide portion 340 and the hand of the user P1 are spaced apart from each other by a predetermined interval. Furthermore, the information processing apparatus 20 may not include the guide portion 340.

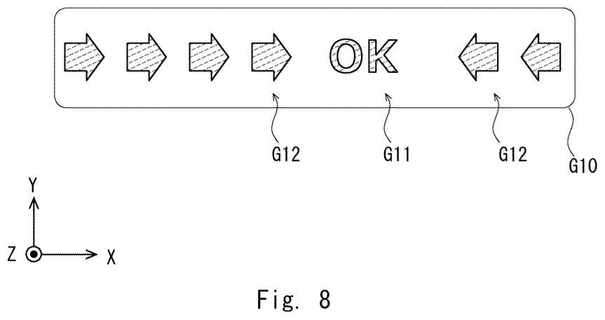

Next, an example of the guidance image will be described with reference to FIG. 8. FIG. 8 is a view illustrating a guidance image G10 projected on the guide portion 340 by the guidance image projection apparatus 320. The guidance image G10 projected by the guidance image projection apparatus 320 includes a first guidance image G11 and a second guidance image G12.

The first guidance image G11 includes a first message to be projected onto the hand existing at the biometric information readable position of the biometric information reading apparatus 330. The biometric information readable position refers to a position where a portion of the hand of the user P1 to be read by the biometric information reading apparatus 330 exists on the readable region A10. The first guidance image G11 illustrated in FIG. 8 includes characters “OK” as the first message. In a case where the first guidance image G11 is projected onto the hand, the user P1 can recognize that the position of the hand is the biometric information readable position.

The second guidance image G12 includes a second message different from the first message projected onto the hand existing at a position separated from the biometric information readable position. The second guidance image G12 illustrated in FIG. 8 includes an arrow symbol as the second message. The arrow is directed to the first image. That is, in a case where the second guidance image G12 is projected onto the hand, the user P1 is prompted to move the hand to the first guidance image G11.

Note that the guidance image G10 may be one in which the image illustrated in FIG. 8 is always projected on the guide portion 340, or may be one that changes according to the position and posture of the hand. Furthermore, in this case, the guide portion 340 is preferably black or the like, and suppresses reflection of light of the guidance image G10. With such a configuration, the user P1 can visually recognize only the image projected on his/her hand and can focus on the message projected on the hand.

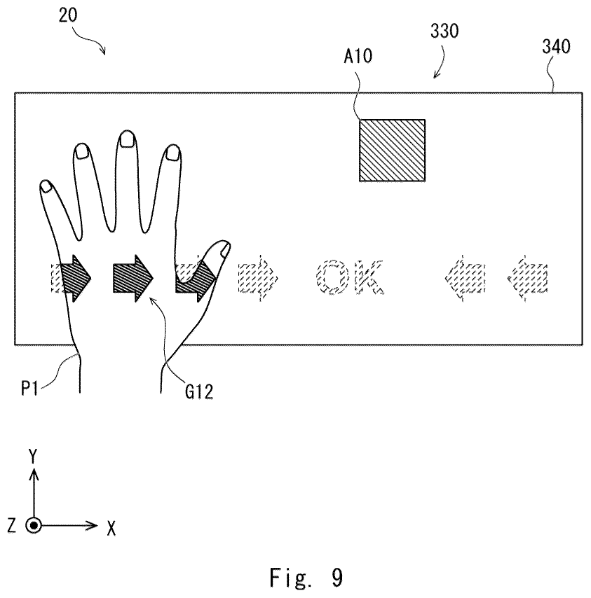

Next, an example of a usage status of the information processing apparatus 20 will be described with reference to FIG. 9. FIG. 9 is a second diagram illustrating a usage example of the information processing apparatus according to the second example embodiment. FIG. 9 is a view of the guide portion 340 and an object on the guide portion 340 observed from above the guide portion 340.

In FIG. 9, the user P1 attempts biometric authentication by putting out his/her hand to the information processing apparatus 20. However, the hand of the user P1 exists at a position away from the readable region A10. The second guidance image G12, which is a guidance image, is projected on the hand of the user P1. The second guidance image G12 projected on the hand of the user P1 is an image of an arrow whose tip is directed to the right in order to prompt the user to move the hand in the right direction (the X-axis plus direction). Therefore, the user P1 moves his/her hand to the right.

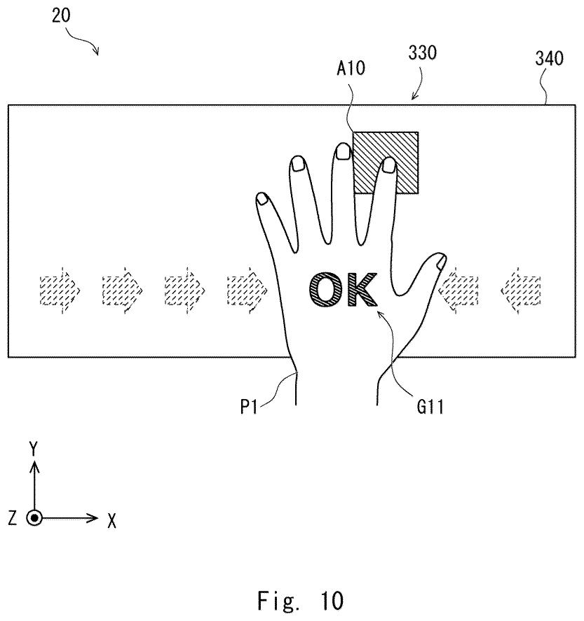

Next, a usage status of the information processing apparatus 20 will be further described with reference to FIG. 10. FIG. 10 is a third diagram illustrating a usage example of the information processing apparatus according to the second example embodiment. FIG. 10 illustrates a situation in which the hand of the user P1 has moved at a later time than the situation illustrated in FIG. 9, for example.

In FIG. 10, the index finger of the left hand of the user P1 enters the readable region A10, and the biometric information reading apparatus 330 can read the biometric information. At this time, the first guidance image G11 is projected on the back of the hand of the user P1. Therefore, the user P1 stops his/her hand. Furthermore, the camera 310 installed above the guide portion 340 images the situation illustrated in FIG. 10. Then, the determination unit 120 that has acquired the projection image captured by the camera 310 from the projection image acquisition unit 110 determines that the biometric information can be read from the received projection image. Further, the control signal output unit 130 outputs a control signal instructing the biometric information reading apparatus 330 to read the biometric information. As a result, the biometric information reading apparatus 330 installed below the guide portion 340 reads the biometric information.

Although the second example embodiment has been described above, the information processing system 1 to the information processing apparatus 20 according to the second example embodiment are not limited to the above-described configurations. For example, the information processing apparatus 20 and the authentication apparatus 400 may directly communicate with each other without a network. Furthermore, the information processing system 1 may include a plurality of information processing apparatuses 20, and a plurality of information processing apparatuses 20 may be connected to one authentication apparatus 400.

In the information processing apparatus 20, the biometric information reading apparatus 330 may have a function of extracting a feature amount of an image read by the biometric information reading apparatus 330. In this case, the information processing apparatus 20 may supply the extracted feature amount of the image to the authentication apparatus 400.

The information processing system 1 described above projects a guidance image onto the hand of the user P1. As a result, the information processing system 1 smoothly acquires the biometric information by the user paying attention to his/her hand. Therefore, the information processing system 1 can smoothly perform authentication. That is, according to the second example embodiment, it is possible to provide an information processing apparatus or the like that prompts the user to perform the authentication operation in an easily understandable manner.

### Third Example Embodiment

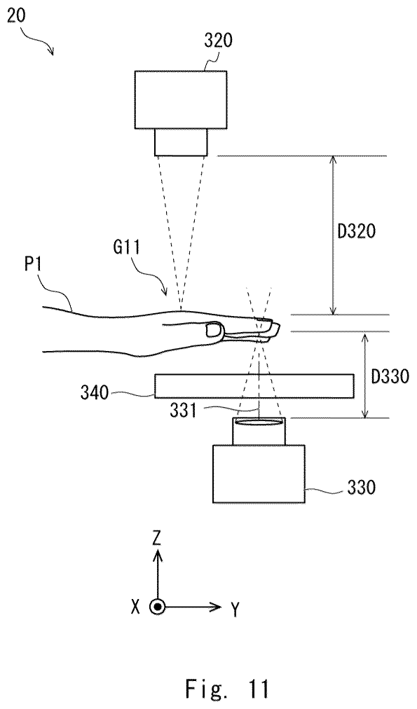

Next, the third example embodiment will be further described with reference to FIG. 11. In the third example embodiment, a function is further added to the determination unit 120 of the second example embodiment. FIG. 11 illustrates an example of a case where the hand of the user P1 is guided to a position away from the guide portion 340.

In FIG. 11, the guidance image projection apparatus 320 projects a guidance image onto the back of the hand of the user P1. At this time, the guidance image projection apparatus 320 is configured such that the guidance image is clearly formed on the back of the hand when the guidance image projection apparatus 320 and the back of the hand are at the distance D320. That is, in a case where the back of the hand moves downward and exists at a position farther than the distance D320, the guidance image is an unclear (blurred) image. Similarly, even at a position where the back of the hand moves upward and is closer than the distance D320, the guidance image is an unclear image. Therefore, the user P1 intuitively moves the hand to a position where the guidance image projected on the back of the hand is clearly projected.

Furthermore, in FIG. 11, the biometric information reading apparatus 330 is configured to be able to suitably read the biometric information of the user P1 in a case where the finger of the user P1 is at a distance D330 at a position along the optical axis 331 of the biometric information reading apparatus 330. That is, in this case, the depth of field of the biometric information reading apparatus 330 includes at least the distance D330.

In the above-described configuration, the guidance image projection apparatus 320 forms the first guidance image G11 on the back of the hand in the range of the depth of field of the biometric information reading apparatus 330 with respect to the hand. In other words, the guidance image projection apparatus 320 projects the first guidance image G11 so as not to form an image outside the range of the depth of field of the biometric information reading apparatus 330. With such a configuration, the information processing apparatus 20 can prompt the user P1 to intuitively move the hand to the range of the depth of field of the biometric information reading apparatus 330.

Furthermore, in the above-described configuration, the determination unit 120 determines a state in which the guidance image is formed on the back of the hand of the user P1 as a predetermined condition. With such a configuration, the information processing apparatus 20 guides the hand of the user P1 in a direction along the optical axis of the biometric information reading apparatus 330 with ease of understanding. In addition, the information processing apparatus 20 can instruct the biometric information reading apparatus 330 to read the biometric information with the formation of the guidance image on the hand of the user P1 as a trigger.

Although the third example embodiment has been described above, the information processing system 1 to the information processing apparatus 20 according to the third example embodiment are not limited to the above-described configurations. For example, the information processing apparatus 20 and the authentication apparatus 400 may directly communicate with each other without a network. Furthermore, the information processing system 1 may include a plurality of information processing apparatuses 20, and a plurality of information processing apparatuses 20 may be connected to one authentication apparatus 400.

In the information processing apparatus 20, the biometric information reading apparatus 330 may have a function of extracting a feature amount of an image read by the biometric information reading apparatus 330. In this case, the information processing apparatus 20 may supply the extracted feature amount of the image to the authentication apparatus 400.

The information processing system 1 described above projects a guidance image onto the hand of the user P1. As a result, the information processing system 1 smoothly acquires the biometric information by the user paying attention to his/her hand. Therefore, the information processing system 1 can smoothly perform authentication. That is, according to the third example embodiment, it is possible to provide an information processing apparatus or the like that prompts the user to perform the authentication operation in an easily understandable manner.

### Fourth Example Embodiment

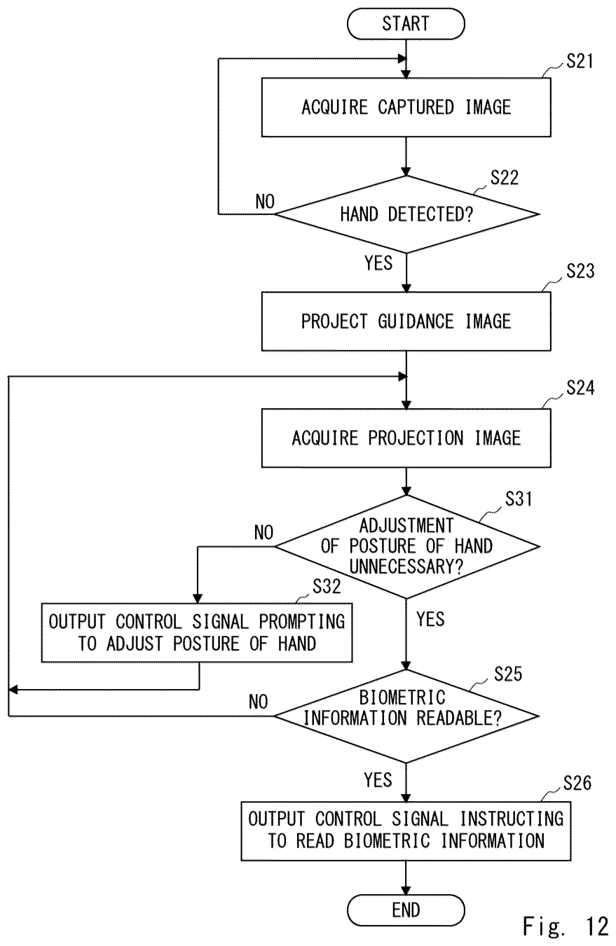

Next, the fourth example embodiment will be described with reference to FIG. 12. The fourth example embodiment is different from the second example embodiment or the third example embodiment in functions of the determination unit 120 and the control signal output unit 130.

The determination unit 120 according to the present example embodiment detects whether the posture of the hand is in a state where the biometric information can be read from the image of the hand included in the projection image. In addition, the determination unit 120 determines that the posture of the hand included in the projection image is in a state where the biometric information can be read as a predetermined condition. Furthermore, in this case, in a case where the posture of the hand does not correspond to the above-described predetermined condition, the control signal output unit 130 outputs a control signal for prompting adjustment of the posture of the hand to, for example, the display apparatus 300. As a result, the display apparatus 300 displays a message prompting adjustment of the posture of the hand.

The processing according to the present example embodiment will be further described with reference to FIG. 12. FIG. 12 is a flowchart illustrating an information processing method according to the fourth example embodiment. The flowchart illustrated in FIG. 12 is different from the flowchart illustrated in FIG. 6 in processing between step S24 and step S25.

In step S24 in FIG. 12, the projection image acquisition unit 110 acquires the projection image captured by the camera 310 (step S24). The projection image acquisition unit 110 supplies the acquired projection image to the determination unit 120.

Next, the determination unit 120 detects the posture of the hand from the received projection image, and determines whether the posture of the hand does not need to be adjusted (step S31). In a case where the posture of the hand included in the projection image is in a state where the biometric information can be read, the determination unit 120 determines that the posture of the hand does not need to be adjusted. In this case (step S31: YES), the information processing apparatus 20 proceeds to step S25. On the other hand, in a case where the posture of the hand included in the projection image is not in a state where the biometric information can be read, the determination unit 120 does not determine that adjustment of the posture of the hand is unnecessary. In this case (step S31: NO), the information processing apparatus 20 proceeds to step S32.

In step S32, the control signal output unit 130 outputs a control signal for prompting adjustment of the posture of the hand (step S32). For example, the control signal output unit 130 causes the display apparatus 300 to display a predetermined call attention message. The call attention message includes, for example, content instructing the posture of the hand. After the control signal output unit 130 outputs the control signal for prompting adjustment of the posture of the hand, the information processing apparatus 20 returns to step S24 and acquires the projection image again.

In step S25, the determination unit 120 determines whether the biometric information is readable from the received projection image (step S25). When not determining that the biometric information is readable from the received projection image (step S25: NO), the determination unit 120 returns to step S24. When determining that the biometric information is readable from the received projection image (step S25: YES), the determination unit 120 proceeds to step S26.

Although the information processing method according to the present example embodiment has been described above, the processing executed by the information processing apparatus 20 according to the present example embodiment is not limited to the above-described contents. For example, the determination unit 120 may detect whether the hand included in the projection image is the right hand or the left hand. In addition, the determination unit 120 may perform the above-described determination under a predetermined condition that one of the preset right hand and left hand is detected. Furthermore, in this case, in a case where the image of the hand does not correspond to the predetermined condition, the control signal output unit 130 outputs a control signal for prompting presentation of the opposite hand to the display apparatus 300, for example.

More specifically, for example, in a case where reading the biometric information from the index finger of the left hand by the information processing apparatus 20 is set as a condition for reading the biometric information, it is assumed that the hand detected by the determination unit 120 is the right hand. In this case, the determination unit 120 supplies a signal indicating that the detected hand does not meet the predetermined condition to the control signal output unit 130 as a determination result. When receiving the above-mentioned signal from the determination unit 120, the control signal output unit 130 outputs a control signal for outputting a message for prompting the display apparatus 300 to present an opposite hand.

As described above, in a case where the hand presented by the user P1 does not match the condition for reading the biometric information, the information processing apparatus 20 according to the present example embodiment prompts the user P1 to correct or adjust the hand. Therefore, the fourth example embodiment can provide an information processing apparatus or the like that prompts the user to perform the authentication operation in an easily understandable manner and appropriately prompts the user to perform correction or adjustment.

### Fifth Example Embodiment

Next, a fifth example embodiment will be described. The fifth example embodiment is different from the above-described example embodiment in that a guidance image can be changed.

The determination unit 120 according to the present example embodiment calculates the size ratio between the hand included in the projection image and the projection image projected on the hand. Then, the determination unit 120 determines that the calculated size ratio is within a predetermined range as a predetermined condition. In this case, when the size ratio does not correspond to the predetermined condition, the control signal output unit 130 outputs a control signal for changing the size of the projection image to the guidance image projection apparatus 320.

Furthermore, the guidance image projection apparatus 320 according to the present example embodiment may include a tracking mechanism that projects a guidance image following the user's hand. In this case, the guidance image projection apparatus 320 may be configured to project a guidance image onto the position of the hand detected by the determination unit 120. The tracking mechanism may include a driving mechanism that changes the orientation of the guidance image projection apparatus 320 itself. In addition, instead of the above-described drive mechanism, the following mechanism may project a guidance image at a pixel position corresponding to the position of the hand in a predetermined image projection range included in the guidance image projection apparatus 320. In this case, the control signal output unit 130 receives the determination from the determination unit 120, and outputs a control signal for outputting a guidance image to the guidance image projection apparatus 320 according to the received determination result.

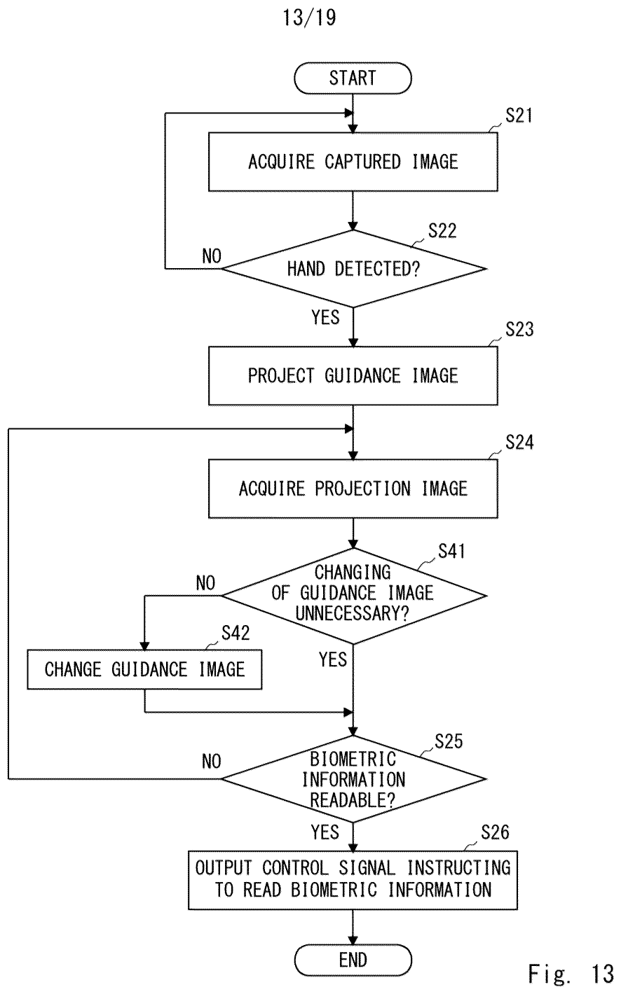

FIG. 13 is a flowchart illustrating an information processing method according to the fifth example embodiment. The flowchart illustrated in FIG. 13 is different from the flowchart illustrated in FIG. 6 in that step S41 and step S42 are provided between step S24 and step S25.

After acquiring the projection image in step S24 according to the present example embodiment, the determination unit 120 determines whether it is necessary to change the guidance image from the received projection image. For example, the determination unit 120 measures the size of the image of the hand included in the projection image and the size of the guidance image projected on the hand, and calculates a size ratio thereof. Then, when the calculated size ratio is within the predetermined range, the determination unit 120 determines that there is no need to change the guidance image. In this case (step S41: YES), the information processing apparatus 20 proceeds to step S24 without changing the guidance image. On the other hand, when the calculated size ratio is out of the predetermined range, the determination unit 120 does not determine that there is no need to change the guidance image. In this case (step S41: NO), the information processing apparatus 20 proceeds to step S42.

In step S42, the control signal output unit 130 generates and outputs a control signal instructing the guidance image projection apparatus 320 to change the guidance image according to the determination result. Here, for example, the control signal output unit 130 calculates or selects the size of the guidance image according to the size ratio calculated by the determination unit 120 such that the size ratio falls within a predetermined range, and generates and outputs a control signal for projecting the guidance image according to the calculation or the selection. Through such processing, the information processing apparatus 20 changes the guidance image (step S42). When the guidance image is changed, the information processing apparatus 20 proceeds to step S25.

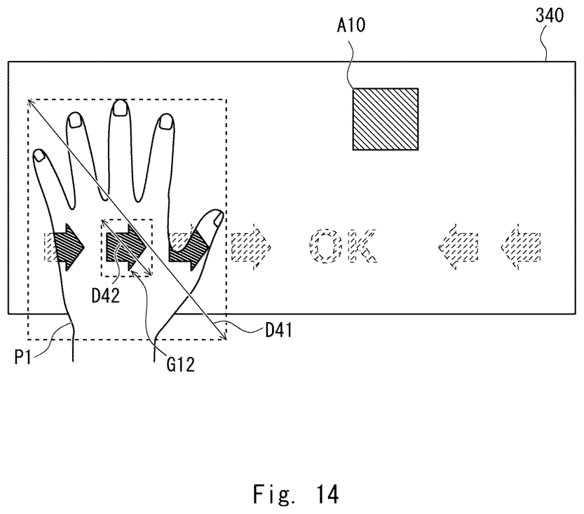

FIG. 14 is a first diagram illustrating a usage example of the information processing apparatus according to the fifth example embodiment. FIG. 14 is a projection image captured by the camera 310, and includes a hand of the user P1 and the second guidance image G12 projected on the hand of the user P1. Here, upon receiving the image of FIG. 14 from the projection image acquisition unit 110, the determination unit 120 according to the present example embodiment measures the size of the hand and the image of the second guidance image G12 projected on the hand.

When measuring the size of the hand, the determination unit 120 first recognizes an image of the hand. As a method of recognizing the image of the hand by the determination unit 120, a general object detection technique can be applied. That is, the determination unit 120 recognizes the image of the hand included in the projection image using, for example, histograms of oriented gradient (HOG), a support vector machine, a neural network with convolution processing, or the like. Next, the length of the diagonal line is measured from the image of the recognized hand. In the example illustrated in FIG. 14, the diagonal line of the hand is a length D41.

Next, the determination unit 120 recognizes the guidance image projected on the hand, and measures the size of the recognized guidance image. In the case of the example illustrated in FIG. 14, a diagonal line of the second guidance image G12 is a length D42.

Next, the determination unit 120 calculates a size ratio between the diagonal line of the hand and the diagonal line of the guidance image. For example, specifically, the determination unit 120 sets a value obtained by dividing the length D41 of the diagonal line of the hand by the length D42 of the diagonal line of the guidance image as the size ratio. Then, the determination unit 120 compares the size ratio with a predetermined numerical range stored in advance. As a result, the determination unit 120 determines whether it is inside.

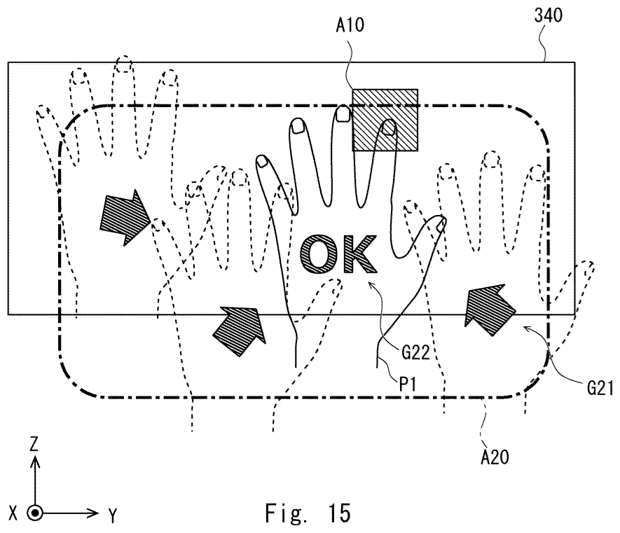

Next, an example in which the guidance image projected by the guidance image projection apparatus 320 fluctuates will be described with reference to FIG. 15. FIG. 15 is a second diagram illustrating a usage example of the information processing apparatus according to the fourth example embodiment.

FIG. 15 shows the hand of the user P1 in a plurality of different locations. The three hands indicated by dotted lines in the hand of the user P1 illustrated in FIG. 15 have different positions in the X direction and the Y direction. In addition, in FIG. 15, a guidance image is projected on the back of each hand. Each of the guidance images projected on the hand located at a position away from the readable region A10 is an arrow, and the leading end faces the readable region A10. As described above, the guidance image projection apparatus 320 according to the present example embodiment can project the guidance image for guiding the hand of the user P1 to the readable region A10 on the hand of the user P1 within the range of the image projectable region A20.

In this manner, the information processing apparatus 20 according to the present example embodiment can flexibly display the guidance image in accordance with the size and position of the hand of the user P1. Therefore, the fourth example embodiment can provide an information processing apparatus or the like that flexibly prompts the authentication operation in an easily understandable manner for the user.

### Sixth Example Embodiment

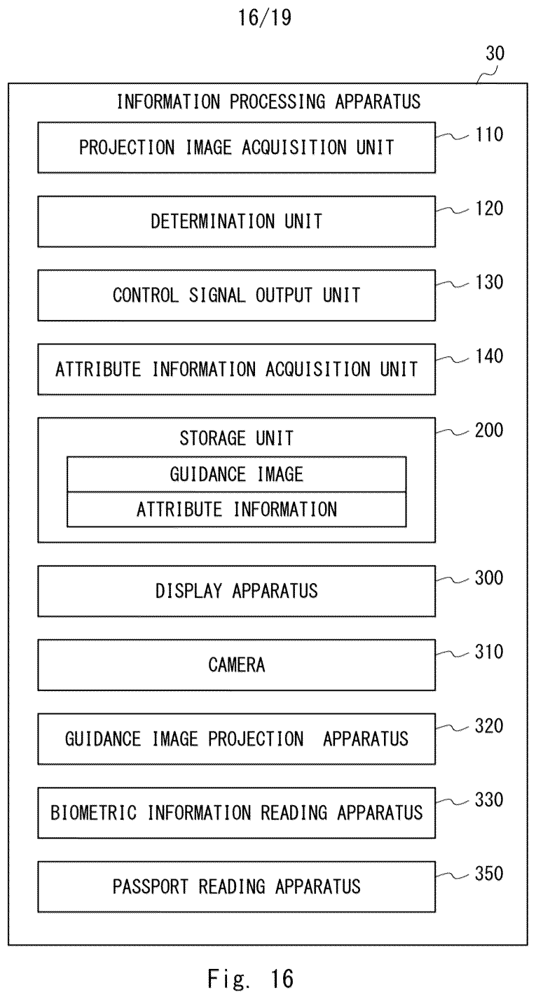

Next, the sixth example embodiment will be described with reference to FIG. 16. FIG. 16 is a block diagram illustrating a configuration of an information processing apparatus 30 according to the sixth example embodiment. The information processing apparatus 30 according to the present example embodiment is different from the above-described example embodiment in that it includes an attribute information acquisition unit 140 and a passport reading apparatus 350.

The attribute information acquisition unit 140 included in the information processing apparatus 30 acquires the attribute information of the user P1 who reads the biometric information. The attribute information includes at least one of nationality, address, residence, travel history, age, and gender of the user P1. The attribute information acquisition unit 140 acquires the attribute information from the information of the passport read by the passport reading apparatus 350.

The control signal output unit 130 according to the present example embodiment outputs a control signal for outputting a guidance image corresponding to the attribute information acquired by the attribute information acquisition unit 140 to the guidance image projection apparatus 320. At this time, the control signal output unit 130 collates the attribute information acquired by the attribute information acquisition unit 140 with the attribute information stored in the storage unit 200. Then, the control signal output unit 130 reads the guidance image corresponding to the attribute information acquired by the attribute information acquisition unit 140 from the storage unit 200, and outputs a control signal for causing the guidance image projection apparatus 320 to project the read guidance image.

The storage unit 200 according to the present example embodiment stores a plurality of guidance images and attribute information corresponding to each of the guidance images in association with each other. In the guidance image in the present example embodiment, for example, the displayed language is different according to the attribute information. As a result, a language familiar to each user can be used for the guidance image. Similarly, in the guidance image in the present example embodiment, displayed symbols and the like can also be set according to the attribute information. Therefore, the information processing apparatus 30 can display a guidance image that is easy for each user to understand.

The passport reading apparatus 350 according to the present example embodiment reads predetermined information written in the passport of the user P1. The passport reading apparatus 350 is, for example, a scanner including an imaging element. In this case, the passport reading apparatus 350 images the passport presented by the user P1, identifies characters from the captured image, and supplies the identified character information to the attribute information acquisition unit 140. Note that, in a case where the attribute information is written in the passport of the user P1 by a method other than characters, the passport reading apparatus 350 may have a means for reading the attribute information instead of the scanner. Specifically, for example, the passport reading apparatus 350 may be a barcode reader, a magnetic information reading apparatus, a near field communication apparatus, or the like.

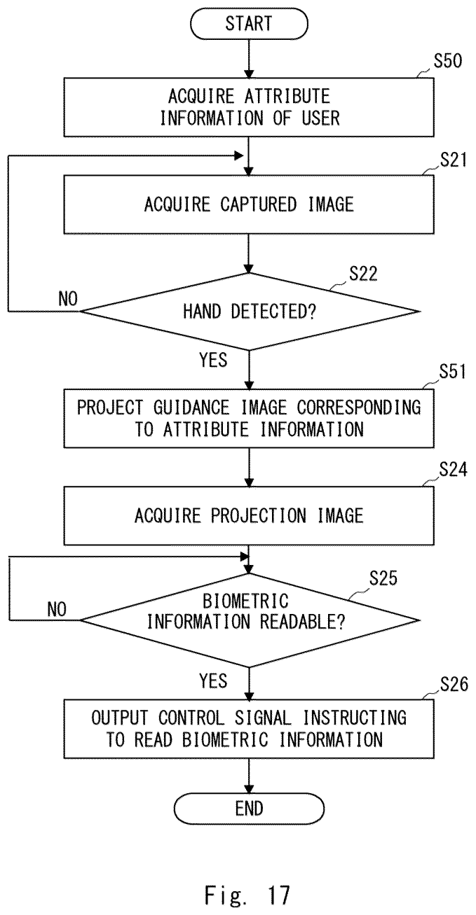

Next, processing of the information processing apparatus 30 will be described with reference to FIG. 17. FIG. 17 is a flowchart illustrating an information processing method according to the sixth example embodiment. The flowchart illustrated in FIG. 17 is different from the flowchart illustrated in FIG. 6 in that step S50 is provided before step S21 and step S51 is provided instead of step S23.

First, the information processing apparatus 30 acquires attribute information of the user (step S50). More specifically, the attribute information acquisition unit 140 of the information processing apparatus 30 extracts predetermined attribute information from the information on the passport read by the passport reading apparatus 350.

Next, the information processing apparatus 30 executes steps S21 and S22 to attempt to detect the hand of the user P1. In a case where the hand of the user P1 is detected in step S22 (step S22: YES), the information processing apparatus 30 projects a guidance image corresponding to the attribute information (step S51). More specifically, the control signal output unit 130 collates the attribute information acquired by the attribute information acquisition unit 140 with the attribute information stored in the storage unit 200 and the guidance image associated therewith. Then, the control signal output unit 130 selects a guidance image corresponding to the acquired attribute information. Further, the control signal output unit 130 outputs a control signal instructing to project the selected guidance image to the guidance image projection apparatus 320. Since the subsequent processing is similar to that in FIG. 6, the description thereof is omitted here.

The sixth example embodiment has been described above. Note that, in the above-described flowchart, step S50 may be before step S51. That is, step S50 may be performed after step S21 or step S22, or may be performed in parallel. The information processing apparatus 30 may also use the attribute information acquired by the attribute information acquisition unit 140 for the message to be displayed on the display apparatus. As a result, the information processing apparatus 30 can display various messages and the like in an easily understandable manner for the user. In addition, the above-described example embodiments can be appropriately combined.

With the above-described configuration, the information processing system including the information processing apparatus 30 can smoothly perform authentication. That is, according to the sixth example embodiment, it is possible to provide an information processing apparatus or the like that prompts the user to perform the authentication operation in an easily understandable manner.

### Seventh Example Embodiment

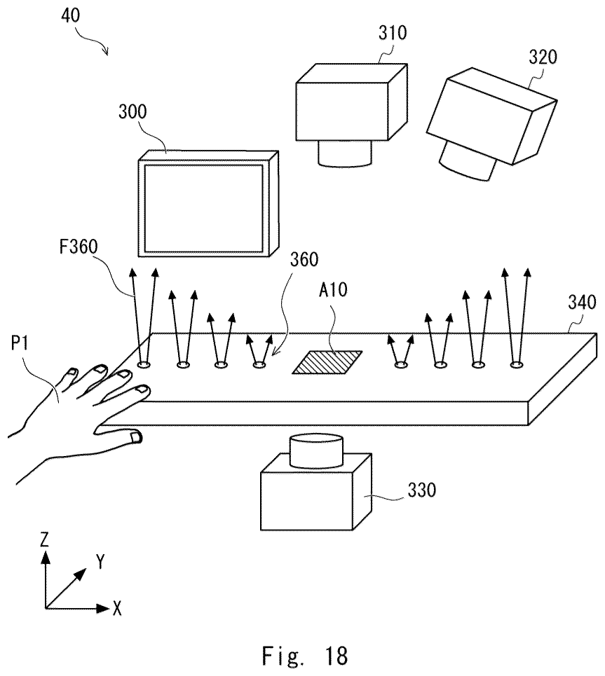

Next, a seventh example embodiment will be described. FIG. 18 is a diagram illustrating a configuration of an information processing apparatus 40 according to the seventh example embodiment. The present example embodiment is different from the above-described example embodiment in including a tactile stimulation means corresponding to a guidance image.

The information processing apparatus 40 includes a tactile stimulation apparatus 360. The tactile stimulation apparatus 360 stimulates the tactile sensation of the hand with respect to the hand on which the guidance image is projected. In addition, the tactile stimulation apparatus 360 is set such that the stimulation to the tactile sense of the hand becomes weaker as the distance to the biometric information reading apparatus becomes relatively shorter, and the stimulation to the tactile sense of the hand becomes stronger as the distance to the biometric information reading apparatus becomes relatively longer.

In the tactile stimulation apparatus 360 illustrated in FIG. 18, a plurality of ejection ports are provided in the guide portion 340 as a specific means for stimulating a tactile sensation of a hand. The ejection port ejects air toward the presented user's hand. The flow velocity of the air blown out from each of the ejection ports is set depending on the disposed position. For example, the flow velocity of the air blown out from each of the ejection ports is set to be slower as the distance from the biometric information reading apparatus 330 is relatively shorter, and to be faster as the distance from the biometric information reading apparatus 330 is relatively longer. In FIG. 18, arrows extending upward from the ejection ports of the tactile stimulation apparatus 360 schematically indicate flow velocities F360 of air ejected from the ejection ports. As illustrated in the figure, the flow velocity F360 is shorter toward the readable region A10. Further, the flow velocity F360 is longer as it is farther from the readable region A10.

The tactile stimulation apparatus 360 associates the flow velocity and the air blowing direction with the readable region A10. Specifically, the tactile stimulation apparatus 360 illustrated in FIG. 18 is set so that air blown out from the tactile stimulation apparatus 360 does not hit a hand when the hand is located in the readable region A10.

With the above-described configuration, the information processing apparatus 40 according to the present example embodiment can guide a hand to the biometric information readable position not only for the user who can view the guidance image but also for the user who has difficulty in viewing the guidance image or the user who cannot view the guidance image.

Although the seventh example embodiment has been described above, the information processing apparatus 40 according to the seventh example embodiment is not limited to the above-described configuration. For example, the tactile stimulation apparatus 360 may be arranged at any position on a plane or may be arranged three-dimensionally instead of the linear ejection port as illustrated in FIG. 18. The tactile stimulation apparatus 360 may have an object that touches the hand of the user P instead of the air. The object to be touched by a hand may be metal, resin, wood, organic substance, or the like. The object to be touched by the hand may be stimulated by vibration. The object to be touched by the hand may be stimulated by texture.

As described above, according to the present example embodiment, it is possible to provide an information processing apparatus or the like that prompts an authentication operation in an easily understandable manner for various users.

### Hardware Configuration Example

Hereinafter, a case where each functional configuration of the determination apparatus in the disclosure is realized by a combination of hardware and software will be described.

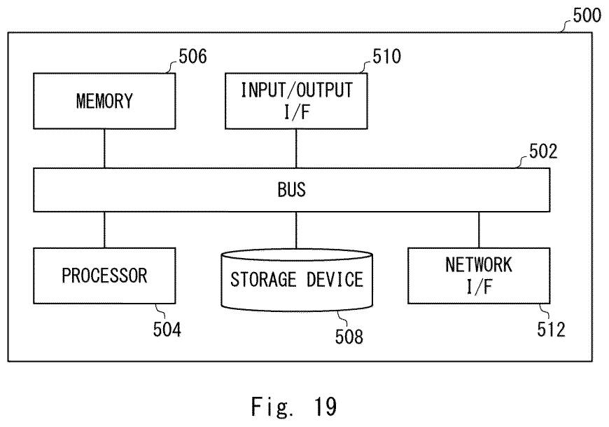

FIG. 19 is a block diagram illustrating the hardware configuration of a computer. The management apparatus according to the disclosure can implement the above-described functions by a computer 500 including the hardware configuration illustrated in the drawings. The computer 500 may be a portable computer such as a smartphone or a tablet terminal or may be a stationary computer such as a PC. The computer 500 may be a dedicated computer designed to implement each apparatus, or may be a general-purpose computer. The computer 500 can realize the corresponding function by installing a predetermined program.

The computer 500 includes a bus 502, a processor 504, a memory 506, a storage device 508, an input/output interface 510 (the interface will also be referred to as an I/F (interface)), and a network interface 512. The bus 502 is a data transmission path for the processor 504, the memory 506, the storage device 508, the input/output interface 510, and the network interface 512 to transmit and receive data to and from each other. However, the method of connecting the processor 504 and the like to each other is not limited to the bus connection.

The processor 504 is various processors such as a CPU, a GPU, or an FPGA. The memory 506 is a primary storage device realized by using a random access memory (RAM) or the like.

The storage device 508 is an auxiliary storage device implemented by using a hard disk, an SSD, a memory card, a read only memory (ROM), or the like. The storage device 508 stores a program for realizing a predetermined function. The processor 504 reads the program to the memory 506 and executes the program to realize each functional component unit of each apparatus.

The input/output interface 510 is an interface connecting the computer 500 and an input/output apparatus. For example, an input apparatus such as a keyboard and an output apparatus such as a display apparatus are connected to the input/output interface 510.

The network interface 512 is an interface connecting the computer 500 to a network.

Although the example of the hardware configuration in the disclosure has been described above, the above-described example embodiment is not limited thereto. The disclosure can also be implemented by causing a processor to execute a computer program.

In the above-described example, the program includes a group of instructions (or software code) for causing a computer to execute one or more functions described in the example embodiments when being read by the computer. The program may be stored in a non-transitory computer-readable medium or a tangible storage medium. As an example and not by way of limitation, a computer-readable medium or tangible storage medium includes a random-access memory (RAM), a read-only memory (ROM), a flash memory, a solid-state drive (SSD) or other memory technology, a CD-ROM, a digital versatile disc (DVD), a Blu-ray (registered trademark) disk or other optical disk storage, a magnetic cassette, a magnetic tape, a magnetic disk storage, or other magnetic storage devices. The program may be transmitted on a transitory computer-readable medium or a communication medium. As an example and not by way of limitation, transitory computer-readable medium or communication medium include electrical, optical, acoustic, or other forms of propagated signals.

Although the invention of the present application has been described above with reference to the example embodiments, the invention of the present application is not limited to the above. Various modifications that can be understood by those skilled in the art can be made to the configuration and details of the invention of the present application within the scope of the invention.

Some or all of the above-described example embodiments may be described as in the following Supplementary Notes, but are not limited to the following Supplementary Notes.

### Supplementary Note 1

An information processing apparatus including:
a projection image acquisition unit that acquires a projection image obtained by projecting a predetermined guidance image onto a hand of a user presented on a biometric information reading apparatus configured to read predetermined biometric information from the user's hand;
a determination unit that determines whether the guidance image and the image of the hand included in the projection image correspond to a predetermined condition; and
a control signal output unit that outputs a predetermined control signal to the biometric information reading apparatus based on the determination.

### Supplementary Note 2

The information processing apparatus according to supplementary note 1, in which
the determination unit performs the determination under the predetermined condition that a trigger image indicating start of reading biometric information in the guidance image is projected on the hand, and
the control signal output unit outputs the control signal for starting reading to the biometric information reading apparatus when the projection image corresponds to the predetermined condition.

### Supplementary Note 3

The information processing apparatus according to supplementary note 1, in which
the determination unit performs the determination under the predetermined condition that the posture of the hand included in the projection image is in a state where biometric information is readable, and
the control signal output unit outputs the control signal for prompting adjustment of the posture of the hand when the posture of the hand does not correspond to the predetermined condition.

### Supplementary Note 4

The information processing apparatus according to supplementary note 1, in which
the determination unit detects whether the hand included in the projection image is a right hand or a left hand, and performs the determination under the predetermined condition that one of the right hand or the left hand set in advance is detected, and
the control signal output unit outputs the control signal for prompting presentation of the opposite hand when the image of the hand does not correspond to the predetermined condition.

### Supplementary Note 5

The information processing apparatus according to any one of supplementary notes 2 to 4, further including: the biometric information reading apparatus that is provided at a position where biometric information can be read from a finger of the hand corresponding to the predetermined condition to perform a biometric information reading operation according to the control signal.

### Supplementary Note 6

An information processing system including:
the information processing apparatus according to supplementary note 5; and
an authentication apparatus that acquires biometric information read by the biometric information reading apparatus, authenticates the acquired biometric information, and supplies a result of the authentication to the information processing apparatus.

### Supplementary Note 7

The information processing apparatus according to supplementary note 1, further including: a guidance image projection apparatus that projects a first guidance image including a first message to be projected onto the hand existing at a biometric information readable position of the biometric information reading apparatus and a second guidance image including a second message different from the first message to be projected onto the hand existing at a position separated from the biometric information readable position.

### Supplementary Note 8

The information processing apparatus according to supplementary note 7, in which the guidance image projection apparatus forms an image in a range of a depth of field of the biometric information reading apparatus with respect to the hand, and projects the first guidance image so as not to form an image outside the range of the depth of field.

### Supplementary Note 9

The information processing apparatus according to supplementary note 8, in which the determination unit determines that the first guidance image is formed on the hand as the predetermined condition.

### Supplementary Note 10

The information processing apparatus according to supplementary note 7, in which
the determination unit calculates a size ratio between the hand included in the projection image and the projection image, and performs the determination under the predetermined condition that the size ratio is within a predetermined range, and
the control signal output unit outputs the control signal for changing the size of the projection image to the guidance image projection apparatus when the size ratio does not correspond to the predetermined condition.

### Supplementary Note 11

The information processing apparatus according to supplementary note 7, in which
the guidance image projection apparatus includes a following mechanism that projects the guidance image following the user's hand, and
the control signal output unit outputs a control signal for causing the guidance image projection apparatus to output the guidance image according to the position of the hand based on the determination.

### Supplementary Note 12

The information processing apparatus according to any one of supplementary notes 7 to 11, further including:
an attribute information acquisition unit that acquires attribute information of the user, in which
the control signal output unit outputs the control signal for outputting the guidance image corresponding to the acquired attribute information to the guidance image projection apparatus.

### Supplementary Note 13

The information processing apparatus according to supplementary note 12, further including: the biometric information reading apparatus that is provided at a position where biometric information can be read from a finger of the hand corresponding to the predetermined condition to perform a biometric information reading operation according to the control signal.

### Supplementary Note 14

An information processing system including:
the information processing apparatus according to supplementary note 12; and
an authentication apparatus that acquires biometric information read by the biometric information reading apparatus, authenticates the acquired biometric information, and supplies a result of the authentication to the information processing apparatus.

### Supplementary Note 15

An information processing method for causing a computer to execute:
acquiring a projection image obtained by projecting a predetermined guidance image onto a hand of a user presented on a biometric information reading apparatus;
determining whether the guidance image and the image of the hand included in the projection image correspond to a predetermined condition; and
outputting a predetermined control signal to the biometric information reading apparatus based on the determination.

### Supplementary Note 16

A program for causing a computer to execute an information processing method including:
acquiring a projection image obtained by projecting a predetermined guidance image onto a hand of a user presented on a biometric information reading apparatus;
determining whether the guidance image and the image of the hand included in the projection image correspond to a predetermined condition; and
outputting a predetermined control signal to the biometric information reading apparatus based on the determination.

### Supplementary Note 17

The information processing system according to supplementary note 14, further including:
a tactile stimulation apparatus that stimulates a tactile sensation of the hand with respect to the hand on which the guidance image is projected, in which
the stimulation to the tactile sense of the hand is set so as to be weaker as the distance to the biometric information reading apparatus is relatively shorter, and so as to be stronger as the distance to the biometric information reading apparatus is relatively longer.

Although the invention of the present application has been described above with reference to the example embodiments, the invention of the present application is not limited to the above. Various modifications that can be understood by those skilled in the art can be made to the configuration and details of the invention of the present application within the scope of the invention.

This application claims priority based on Japanese Patent Application No. 2022-085696 filed on May 26, 2022, the disclosure of which is incorporated herein in its entirety.

### INDUSTRIAL APPLICABILITY

The disclosure can be used, for example, as a system that performs biometric authentication of a user who uses an airport, a port, or the like.

### REFERENCE SIGNS LIST

1 INFORMATION PROCESSING SYSTEM
10 INFORMATION PROCESSING APPARATUS
20 INFORMATION PROCESSING APPARATUS
30 INFORMATION PROCESSING APPARATUS
40 INFORMATION PROCESSING APPARATUS
110 PROJECTION IMAGE ACQUISITION UNIT
120 DETERMINATION UNIT
130 CONTROL SIGNAL OUTPUT UNIT
140 ATTRIBUTE INFORMATION ACQUISITION UNIT
200 STORAGE UNIT
300 DISPLAY APPARATUS
310 CAMERA
320 GUIDANCE IMAGE PROJECTION APPARATUS
330 BIOMETRIC INFORMATION READING APPARATUS
340 GUIDE
350 PASSPORT READING APPARATUS
360 TACTILE STIMULATION APPARATUS
400 AUTHENTICATION APPARATUS
410 AUTHENTICATION STORAGE UNIT
420 FEATURE IMAGE EXTRACTION UNIT
430 FEATURE POINT EXTRACTION UNIT
440 REGISTRATION UNIT
450 AUTHENTICATION UNIT
500 COMPUTER
504 PROCESSOR
506 MEMORY
508 STORAGE DEVICE
510 INPUT/OUTPUT INTERFACE
512 NETWORK INTERFACE
A10 READABLE REGION
G10 GUIDANCE IMAGE
G11 FIRST GUIDANCE IMAGE
G12 SECOND GUIDANCE IMAGE
N1 NETWORK
P1 USER

What is claimed is:

1. An information processing apparatus comprising:
at least one memory storing computer-executable instructions; and
at least one processor configured to access the at least one memory and execute the computer-executable instructions to:
acquire a projection image obtained by projecting a predetermined guidance image onto a hand of a user presented on a biometric information reading apparatus configured to read predetermined biometric information from the user's hand;
determine whether the guidance image and the image of the hand included in the projection image correspond to a predetermined condition; and
output a predetermined control signal to the biometric information reading apparatus based on the determination.

2. The information processing apparatus according to claim 1, wherein the at least one processor is further configured to execute the instructions to:
perform the determination under the predetermined condition that a trigger image indicating start of reading biometric information in the guidance image is projected on the hand, and
output the control signal for starting reading to the biometric information reading apparatus when the projection image corresponds to the predetermined condition.

3. The information processing apparatus according to claim 1, wherein the at least one processor is further configured to execute the instructions to:
perform the determination under the predetermined condition that the posture of the hand included in the projection image is in a state where biometric information is readable, and
output the control signal for prompting adjustment of the posture of the hand when the posture of the hand does not correspond to the predetermined condition.

4. The information processing apparatus according to claim 1, wherein the at least one processor is further configured to execute the instructions to:
detect whether the hand included in the projection image is a right hand or a left hand, and perform the determination under the predetermined condition that one of the right hand or the left hand set in advance is detected, and
output the control signal for prompting presentation of the opposite hand when the image of the hand does not correspond to the predetermined condition.

5. The information processing apparatus according to claim 2, further comprising: the biometric information reading apparatus that is provided at a position where biometric information can be read from a finger of the hand corresponding to the predetermined condition to perform a biometric information reading operation according to the control signal.

6. An information processing system comprising:
the information processing apparatus according to claim 5; and
an authentication apparatus that acquires biometric information read by the biometric information reading apparatus, authenticates the acquired biometric information, and supplies a result of the authentication to the information processing apparatus.

7. The information processing system according to claim 6, further comprising:
a tactile stimulation apparatus that stimulates a tactile sensation of the hand with respect to the hand on which the guidance image is projected, wherein
the stimulation to the tactile sense of the hand is set so as to be weaker as the distance to the biometric information reading apparatus is relatively shorter, and so as to be stronger as the distance to the biometric information reading apparatus is relatively longer.

8. The information processing apparatus according to claim 1, further comprising: a guidance image projection apparatus that projects a first guidance image including a first message to be projected onto the hand existing at a biometric information readable position of the biometric information reading apparatus and a second guidance image including a second message different from the first message to be projected onto the hand existing at a position separated from the biometric information readable position.

9. The information processing apparatus according to claim 8, wherein the guidance image projection apparatus forms an image in a range of a depth of field of the biometric information reading apparatus with respect to the hand, and projects the first guidance image so as not to form an image outside the range of the depth of field.

10. The information processing apparatus according to claim 9, wherein the at least one processor is further configured to execute the instructions to:
determine that the first guidance image is formed on the hand as the predetermined condition.

11. The information processing apparatus according to claim 8, wherein the at least one processor is further configured to execute the instructions to:
calculate a size ratio between the hand included in the projection image and the projection image, and perform the determination under the predetermined condition that the size ratio is within a predetermined range, and
output the control signal for changing the size of the projection image to the guidance image projection apparatus when the size ratio does not correspond to the predetermined condition.

12. The information processing apparatus according to claim 8, wherein
the guidance image projection apparatus includes a following mechanism that projects the guidance image following the user's hand, and
the at least one processor is further configured to execute the instructions to output a control signal for causing the guidance image projection apparatus to output the guidance image according to the position of the hand based on the determination.

13. The information processing apparatus according to claim 8, wherein the at least one processor is further configured to execute the instructions to:
acquire attribute information of the user, and
output the control signal for outputting the guidance image corresponding to the acquired attribute information to the guidance image projection apparatus.

14. The information processing apparatus according to claim 13, further comprising: the biometric information reading apparatus that is provided at a position where biometric information can be read from a finger of the hand corresponding to the predetermined condition to perform a biometric information reading operation according to the control signal.

15. An information processing system comprising:
the information processing apparatus according to claim 13; and
an authentication apparatus that acquires biometric information read by the biometric information reading apparatus, authenticates the acquired biometric information, and supplies a result of the authentication to the information processing apparatus.

16. The information processing system according to claim 15, further comprising:
a tactile stimulation apparatus that stimulates a tactile sensation of the hand with respect to the hand on which the guidance image is projected, wherein
the stimulation to the tactile sense of the hand is set so as to be weaker as the distance to the biometric information reading apparatus is relatively shorter, and so as to be stronger as the distance to the biometric information reading apparatus is relatively longer.

17. An information processing method for causing a computer to execute:
acquiring a projection image obtained by projecting a predetermined guidance image onto a hand of a user presented on a biometric information reading apparatus;
determining whether the guidance image and the image of the hand included in the projection image correspond to a predetermined condition; and
outputting a predetermined control signal to the biometric information reading apparatus based on the determination.

18. A non-transitory computer-readable medium having a program stored thereon, the program causing a computer to execute an information processing method comprising:
acquiring a projection image obtained by projecting a predetermined guidance image onto a hand of a user presented on a biometric information reading apparatus;
determining whether the guidance image and the image of the hand included in the projection image correspond to a predetermined condition; and
outputting a predetermined control signal to the biometric information reading apparatus based on the determination.
